(19) United States
(12) Patent Application Publication
(10) Pub. No.: US 2025/0368587 A1
(43) Pub. Date: Dec. 4, 2025

(54) CONVERTING HYDROGENATION CATALYST DEACTIVATION COMPOUNDS TO NONREACTIVE COMPOUNDS UPSTREAM OF A LIQUID PHASE HYDROGENATION REACTOR

(71) Applicant: Chevron Phillips Chemical Company LP, The Woodlands, TX (US)

(72) Inventors: Steven R. Horlacher, League City, TX (US); Israel Garcia, Kingwood, TX (US); Paul D. Hobson, Humble, TX (US); Daniel M. Hasenberg, Montgomery, TX (US)

(21) Appl. No.: 18/731,269

(22) Filed: Jun. 1, 2024

(51) Int. Cl. C07C 5/10; C07F 15/00

(52) U.S. Cl. CPC C07C 5/10; C07F 15/00; C07C 2601/14

(57) ABSTRACT

Compounds that can deactivate or poison a hydrogenation catalyst are converted to nonreactive compounds that are not reactive with the Group 10 metal(s) of the hydrogenation catalyst. Addition of a sacrificial metal alkyl compound upstream of the hydrogenation reactor produces the nonreactive compounds, which can include nonreactive solid products. In some cases, the nonreactive solid products are removed upstream of the hydrogenation reactor, while in other cases, the nonreactive solid products flow to the hydrogenation reactor and are removed in a cooling loop of the hydrogenation reactor.

### FIELD OF THE DISCLOSURE

This disclosure relates to liquid phase catalyzed hydrogenation of aromatic compounds. More particularly, the disclosure relates to converting hydrogenation catalyst deactivation compounds to nonreactive compounds upstream of a liquid phase hydrogenation reactor.

### BACKGROUND

Aromatic hydrocarbons can be used for the production of cycloalkanes. An aromatic feed stream containing aromatic hydrocarbons and a catalyst feed stream containing a hydrogenation catalyst can be introduced to a hydrogenation reactor, wherein the aromatic hydrocarbons contact the hydrogenation catalyst to produce a cycloalkane product. The aromatic feed stream can also contain catalyst deactivation compounds that undesirably react with the hydrogenation catalyst. The undesirable reactions consume the portion of the hydrogenation catalyst that is reacted with the catalyst deactivation compounds, which effectively deactivates a portion of the hydrogenation catalyst and thus reduces the productivity of the hydrogenation catalyst. Moreover, the reaction product formed by reaction of the catalyst deactivation compounds with the hydrogenation catalyst forms solid particulates, which can deposit on the internal walls of the hydrogenation reactor and equipment associated with the hydrogenation reactor. The solid particulates can also catalyze the production of undesirable products, such as higher molecular weight oils and sludge, which can also contribute to fouling of equipment.

An ongoing need exists to improve hydrogenation catalyst productivity and to reduce or eliminate hydrogenation catalyst deactivation caused by the presence of undesirable chemical compounds in the hydrogenation reactor.

### SUMMARY

Disclosed is a process that can include: introducing a sacrificial metal alkyl compound to a stream or equipment including an aromatic compound and a catalyst deactivation compound, wherein a location of the stream or equipment is upstream of a liquid phase hydrogenation reactor, wherein the location is not in a liquid cooling loop of the liquid phase hydrogenation reactor; and reacting the sacrificial metal alkyl compound with the catalyst deactivation compound in the stream or equipment to form a nonreactive solid product.

Disclosed is a system for liquid phase hydrogenation of an aromatic compound. The system can include: a distillation column operable to separate a crude aromatic mixture including the aromatic compound, a catalyst deactivation compound, and water into an overhead stream including water and a bottoms stream including the aromatic compound and the catalyst deactivation compound; a reboiler operable to heat the bottoms stream to form recycle stream and a purified aromatic hydrocarbon stream; a sacrificial metal alkyl stream including a metal alkyl compound connected to the reboiler, to the recycle stream, to the purified aromatic hydrocarbon stream, or a combination thereof; a liquid phase hydrogenation reactor operable to react the aromatic compound with hydrogen in a presence of a homogeneous hydrogenation catalyst to form a cycloalkane compound; and a liquid cooling loop operable to receive a portion of a liquid reaction medium including liquid phase reaction components and solid particulates and to cool the portion of the liquid reaction medium prior to recycling the cooled liquid reaction medium to the liquid phase hydrogenation reactor.

Other technical features may be readily apparent to one skilled in the art from the following figures, descriptions, and claims.

### BRIEF DESCRIPTION OF THE DRAWINGS

For a more complete understanding of this disclosure, reference is now made to the following description, taken in conjunction with the accompanying drawings, in which:

FIG. 1 illustrates a schematic diagram of a process and system for converting hydrogenation catalyst deactivation compounds to nonreactive compounds upstream of a liquid phase hydrogenation reactor.

FIG. 2 illustrates a schematic diagram of another process and system for converting hydrogenation catalyst deactivation compounds to nonreactive compounds upstream of a liquid phase hydrogenation reactor, where nonreactive solid products are removed upstream of the liquid phase hydrogenation reactor by a separator.

FIG. 3 illustrates a schematic diagram of another process and system for converting hydrogenation catalyst deactivation compounds to nonreactive compounds upstream of a liquid phase hydrogenation reactor, where nonreactive solid products are removed upstream of the liquid phase hydrogenation reactor by a filter.

FIG. 4 illustrates a schematic diagram of another process and system for converting hydrogenation catalyst deactivation compounds to nonreactive compounds upstream of a liquid phase hydrogenation reactor, where nonreactive solid products are removed by a filter connected to a liquid cooling loop of the liquid phase hydrogenation reactor.

### DETAILED DESCRIPTION

“Catalyst deactivation compounds” as used herein includes electronegative chemical species that are highly reactive when they come into contact with Group 10 metals (based on International Union of Pure and Applied Chemistry, IUPAC) such as nickel, palladium, and platinum. Catalyst deactivation compounds disclosed herein can include an oxygen-containing compound, a sulfur-containing compound, a halide-containing compound, a nitrogen-containing compound, a phosphorous-containing compound, a protic solvent, an aprotic solvent, or combinations thereof, which is/are reactive with one or more Group 10 metals. The oxygen-containing compound can include carbon dioxide, oxygen, water, alcohols, aldehydes, ethers, carboxylic acids, ketones, or a combination thereof. The sulfur-containing compound can include hydrogen sulfide, sulfur dioxide, sulfur trioxide, thiol compounds, mercaptan compounds, sulfide compounds, or a combination thereof. The halide-containing compounds can include an organochlorides, (e.g., ethyl chloride), organobromides (e.g., methyl bromide), organofluorides (e.g., fluoromethane), or a combination thereof. The nitrogen-containing compound can include any amine-based compound. The phosphorous-containing compound can include phosphine or an organophosphine. The protic solvent can include any compound having a hydrogen atom bonded to an oxygen atom, a nitrogen atom, or a fluoride atom, such as water, methanol, ethanol, propanol (including isopropanol), n-butanol, acetic acid, formic acid, water, or a combination thereof. The aprotic solvent can include tetrahydrofuran (THF), 2,5-dimethyl THF, acetone, toluene, chlorobenzene, pyridine, acetonitrile, carbon dioxide, or a combination thereof. Some catalyst deactivation compounds may be characterized in more than one group, such as water, which can be characterized as a protic solvent and an oxygen-containing compound.

“Metal alkyl compound” as used herein includes compounds having the formula R₃M, where R is an aliphatic hydrocarbon group having from 1 to 30 carbon atoms and M is aluminum, zinc, lithium, or combinations thereof. In some instances, R is a methyl group, an ethyl group, a propyl group, a butyl group, a hexyl group, an isobutyl group, or combinations thereof. Examples of metal alkyl compound include triethyl aluminum, trimethyl aluminum, triisobutyl aluminum, butyl lithium, diethyl zinc, diethylaluminum ethoxide, or combinations thereof.

“Sacrificial metal alkyl compound” as used herein includes any metal alkyl compound that is added to a stream or equipment containing an aromatic compound and catalyst deactivation compound at a location that is upstream of the hydrogenation reactor and coupled to a feed inlet of the hydrogenation reactor, where the stream or equipment is not in the liquid cooling loop of the hydrogenation reactor. The sacrificial metal alkyl compound is added to react with the catalyst deactivation compound without presence of the hydrogenation catalyst, thus preventing or minimizing detrimental effects such catalyst deactivation compound would have on the hydrogenation catalyst if such catalyst deactivation compound were fed to the hydrogenation reactor. The sacrificial metal alkyl compound is added without an IUPAC Group 10 metal hydrogenation catalyst, to distinguish from aluminum alkyl compounds that can be introduced to the hydrogenation reactor as part of a hydrogenation catalyst system that also contains an IUPAC Group 10 metal.

“Nonreactive compound” and “nonreactive compounds” as used herein refers to compounds that are formed by the reaction of a sacrificial metal alkyl compound with a catalyst deactivation compound, such that the compounds are not reactive with an IUPAC Group 10 metal of a hydrogenation catalyst.

“Nonreactive solid product” and “nonreactive solid products” as used herein refers to nonreactive compounds that are solid particulates in the process fluids of this disclosure. Nonreactive solid products can include metal oxides, metal sulfides, and metal halides, or combinations thereof.

The disclosed systems and processes improve hydrogenation catalyst productivity in context of catalyzed hydrogenation of aromatic hydrocarbons to form cycloalkanes, because catalyst deactivation compounds that can be contained in an aromatic feed stream that is introduced to a hydrogenation reactor are consumed, reacted, or otherwise converted to a nonreactive compound (e.g., non-reactive with the Group 10 metal of the hydrogenation catalyst) at a location that is upstream of the hydrogenation reactor. In some embodiments, the catalyst deactivation compounds, the nonreactive solid products, or both can be removed from system and processes at a location upstream of the hydrogenation reactor.

The conversion of catalyst deactivation compounds to nonreactive compounds improves hydrogenation catalyst productivity in the liquid phase hydrogenation reactor, at least because the hydrogenation catalyst is highly reactive with the catalyst deactivation compounds. When the catalyst deactivation compounds are present in the liquid phase hydrogenation reactor, some of the hydrogenation catalyst reacts with the catalyst deactivation compounds instead of the aromatic hydrocarbon (e.g. benzene), therefore deactivating the portion of hydrogenation catalyst that reacts with the catalyst deactivation compound, resulting in less of the hydrogenation catalyst available to react with aromatic compounds, and requiring more hydrogenation catalyst per unit or aromatic compound. Therefore, conversion of these catalyst deactivation compounds to nonreactive solid compounds upstream of the liquid phase hydrogenation reactor increases hydrogenation catalyst productivity. Conversion of the catalyst deactivation compounds reduces the amount of hydrogenation catalyst required in the liquid phase hydrogenation reactor to achieve compared to the amount of hydrogenation catalyst required when the catalyst deactivation compounds are not converted, to achieve the same conversion of aromatic compound to cycloalkane product.

In aspects where the nonreactive compounds contain nonreactive solid products that are removed, removal of the nonreactive solid products from the process and system as described herein can reduce fouling of the liquid phase hydrogenation reactor, the equipment and streams in the liquid cooling loop associated with the hydrogenation reactor, and/or any other downstream equipment, that may result from undesirable accumulations and deposits of the nonreactive solid products. This is because the nonreactive solid products are no longer in the process and system to deposit onto the internals or walls of the hydrogenation reactor and/or any equipment associated with the liquid cooling loop of the hydrogenation reactor.

Further, any increase in catalyst productivity and/or decrease in equipment fouling increases the run length of the hydrogenation reactions by extending the time between shutdowns, e.g., shutdowns for cleaning or replacing equipment. Also, any increase in catalyst productivity reduces the amount of hydrogenation catalyst required for a given run of hydrogenation reaction.

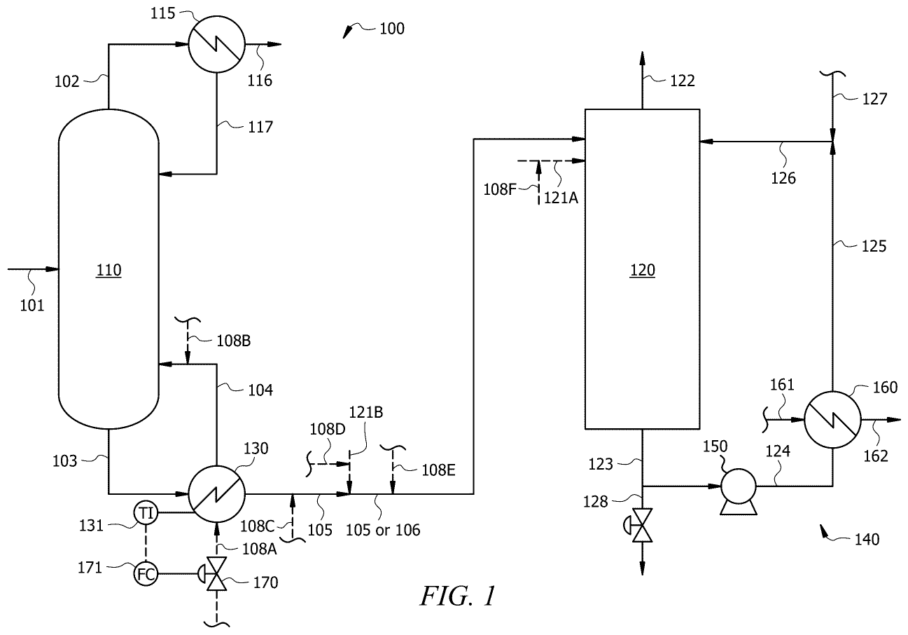

FIG. 1 illustrates a schematic diagram of a process and system 100 for converting hydrogenation catalyst deactivation compounds to nonreactive compounds upstream of a liquid phase hydrogenation reactor. The system 100 includes a distillation column 110 and a liquid phase hydrogenation reactor 120. A reboiler 130 is associated with the distillation column 110 and a liquid cooling loop 140 is associated with the liquid phase hydrogenation reactor 120.

The distillation column 110 comprises a column, an overhead condenser 115, and a reboiler 130 (e.g., kettle). The distillation column internals includes trays, or alternatively, a structured or random packing. The distillation column 110 is operable to receive a crude aromatic mixture from a crude stream 101 and to separate the crude aromatic mixture into a vapor and a liquid. The vapor is recovered in the overhead stream 102 connected to the column, and the liquid is recovered in the bottoms stream 103 connected to the column. The crude aromatic mixture can contain the aromatic compound, catalyst deactivation compounds, and other impurities. The vapor can contain catalyst deactivation compounds, other impurities, and some aromatic compound. The liquid can contain the aromatic compound and catalyst deactivation compounds. In aspects, the crude aromatic mixture contains greater than 20 ppmw of catalyst deactivation compounds based on a total weight of the crude aromatic mixture in the crude stream 101. For example, the crude aromatic mixture can contain 90 wt % or more aromatic compound and 10 wt % or less catalyst deactivation compounds based on a total weight of the crude aromatic mixture.

The overhead condenser 115 can be embodied as a heat exchanger that cools the vapor of overhead stream 102 leaving the column in heat exchange contact with cooling water to either fully or partially condense the vapor into an overhead liquid stream 117. In some embodiments, uncondensed vapor can exit the overhead condenser 115 via stream 116, and the liquid can exit the condenser 115 in liquid stream 117. In other embodiments, such as those where the aromatic compound has an azeotrope with another component in the crude aromatic mixture (such as is the case for benzene as the aromatic compound and water as one of the catalyst deactivation compounds), the aromatic compound (as benzene) can be in the overhead vapor because of the azeotrope. In such cases the aromatic compound and the catalyst deactivation compound can be condensed together in the condenser 115 and separated by techniques known in the art with the aid of this disclosure. For example, because the solubility of benzene in water is very low, when the overhead vapor containing benzene and water is condensed in the condenser 115, the liquid product forms a two-phase mixture of a benzene liquid phase and a water liquid phase than can be separated from one another. The water can be removed from the condenser 115 as a first liquid product in stream 116 and the liquid benzene can be recycled back to the distillation column 110 in a second liquid product in stream 117. In some aspects, stream 116 comprising first liquid product can contain some benzene, for example, less than 10 wt % benzene, less than 5, 4, 3, 2, 1, or 0.5 wt % benzene based on a total weight of the stream 116.

The reboiler 130 can be embodied as a heat exchanger that boils the liquid bottoms from the distillation column 110 forming a vapor that flows in a recycle stream 104 and a liquid that flows in a purified aromatic hydrocarbon stream 105. In some embodiments, the reboiler 130 can be located inside the distillation column 110, and in other embodiments, the reboiler 130 is located externally of the distillation column 110.

In an embodiment, the reboiler 130 can be embodied as a shell and tube heat exchanger. A heating medium, such as steam can flow through the tubes of the reboiler 130, while the process liquid flows on the shell side of the reboiler 130. Alternatively, the reboiler 130 can be embodied as a thermosyphon-type heat exchanger, a jacketed heat exchanger, or a vertical calendria-type evaporator.

The recycle stream 104 is connected to the reboiler 130 and the distillation column 110, and recycles the process vapor from the reboiler 130 back into the distillation column 110. In some embodiments, the process vapor in the recycle stream 104 is only in vapor phase; alternatively, the process vapor in the recycle stream 104 comprises a vapor-liquid mixture. The process vapor in the recycle stream 104 can include a vapor phase of the aromatic compound and a vapor phase of a catalyst deactivation compound. In aspects where the sacrificial metal alkyl compound is introduced into the recycle stream 104, the recycle stream 104 can further include the sacrificial metal alkyl compound, or in cases where there is reaction of the sacrificial metal alkyl compound with catalyst deactivation compound in the recycle stream 104, then the recycle stream 104 additionally or alternatively can include nonreactive compound(s).

The purified aromatic hydrocarbon stream 105 is connected to the reboiler 130. In aspects, the purified aromatic hydrocarbon stream 105 can contain greater than 99.0, 99.1, 99.2, 99.3, 99.4, 99.5, 99.6, 99.7, 99.8, or 99.9 wt % of the aromatic compound based on a total weight of the purified aromatic hydrocarbon stream 105. In aspects, the purified aromatic hydrocarbon stream 105 comprises less than 20, 19, 18, 17, 16, 15, 14, 13, 12, 11, 10, 9, 8, 7, 6, 5, 4, 3, 2, or 1 ppmw catalyst deactivation compound based on a total weight of the purified aromatic hydrocarbon stream 105. In aspects, the purified aromatic hydrocarbon stream 105 can include motive equipment, such as one or more pumps to move the aromatic compound downstream.

It has been found that distillation of the aromatic compound in the distillation column 110 (with reboiler 130) can produce the purified aromatic hydrocarbon stream 105 having less than 20 ppmw of the catalyst deactivation compound based on a total weight of the purified aromatic hydrocarbon stream 105. This can be the concentration of catalyst deactivation compound(s) in the purified aromatic hydrocarbon stream 105 in embodiments where the sacrificial metal alkyl compound is introduced to the process and system 100 downstream of the purified aromatic hydrocarbon stream 105.

The concentration of the catalyst deactivation compound in the purified aromatic hydrocarbon stream 105 can further depend on the location where the sacrificial metal alkyl compound is introduced according to the description herein. For example, in aspects where the sacrificial metal alkyl compound is introduced upstream of the purified aromatic hydrocarbon stream 105, then the purified aromatic hydrocarbon stream 105 can include less than 1 ppmw or even 0 ppmw catalyst deactivation compound based on a total weight of the purified aromatic hydrocarbon stream 105 because the sacrificial metal alkyl compound can be added in an amount sufficient to react with all the catalyst deactivation compound(s) present in at the point of introduction. Thus, the catalyst deactivation compound(s) is/are consumed in reaction with the sacrificial metal alkyl compound upstream of the purified aromatic hydrocarbon stream 105. In such cases, the purified aromatic hydrocarbon stream 105 can contain the nonreactive compounds (including nonreactive solid products) formed by the reaction of the catalyst deactivation compound(s) with the sacrificial metal alkyl compound while containing less than 1 ppmw or even 0 ppmw of the catalyst deactivation compound(s). In aspects where the sacrificial metal alkyl compound is introduced into the purified aromatic hydrocarbon stream 105, then embodiments contemplate that the purified aromatic hydrocarbon stream 105 can have an upstream portion (a portion upstream of the location where the sacrificial metal alkyl compound is introduced) that contains less than 20 ppmw of the catalyst deactivation compound(s) and a downstream portion (a portion downstream of the location where the sacrificial metal alkyl compound is introduced) that contains less than 1 ppmw of the catalyst deactivation compound(s), where the concentration of catalyst deactivation compounds in the upstream portion is greater than the concentration of catalyst deactivation compounds in the downstream portion.

The distillation column 110 can be operated and controlled by measuring a variety of process variables including, but not limited to, the temperature and pressure at the top of the column (e.g., the portion of the column 110 that is near the overhead condenser 115) and the temperature and pressure at the bottom of the column (e.g., the portion of the column 110 that is near the reboiler 130). In addition, the concentration of components can also be measured and controlled. In the case of the separation of benzene and water, crude aromatic mixtures having about 90 wt % or more benzene and about 10 wt % or less water at atmospheric pressure, the crude aromatic mixture forms an azeotrope that boils at a temperature of about 69.4° C., while the pure components benzene and water boil at temperatures of 80.1° C. and 100° C., respectively. In order to separate an aromatic compound from a catalyst deactivation compound with which the aromatic compound is azeotropic, the column 110 is operated in such a way to ensure that vapor at the top of the column 110 (that enters the overhead condenser 115) contains much more catalyst deactivation compound than aromatic compound in the process liquid at the bottom of the column 110 (that enters the reboiler 130) contains much more aromatic compound than catalyst deactivation compound.

In aspects, the pressure in the reboiler 130 can be in a range from 0.17 MPaa (25 psia) to 0.276 MPaa (40 psia); alternatively, in a range of from 0.206 MPaa (30 psia) to 0.241 MPaa (35 psia). In aspects, the temperature in the reboiler 130 is controlled to boil the aromatic compound, e.g., the temperature is at or around the boiling point of the aromatic compound at the pressure in the reboiler 130.

The temperature of the process liquid in the reboiler 130 is indicative of the purity of the process liquid with respect to the aromatic compound. A temperature of the process liquid that is below the boiling point temperature of the aromatic compound in the reboiler 130 indicate that the process liquid is less than pure with respect to the aromatic compound; conversely, a temperature of the process liquid that is at the boiling point temperature of the aromatic compound in the reboiler 130 indicates that the process liquid is more pure with respect to the aromatic compound (e.g., the benzene liquid contains less water). That is, the temperature of the process liquid in the reboiler 130 is indicative of the concentration of the aromatic compound in the process liquid.

In aspects, the temperature of the process liquid in the reboiler 130 can be used to determine and control the flow rate of the sacrificial metal alkyl compound into the process and system 100, and in some cases, to produce a purified aromatic hydrocarbon stream 105 that contains 20, 19, 18, 17, 16, 15, 14, 13, 12, 11, or 10 ppmw or less of catalyst deactivation compounds based on a total weight of the purified aromatic hydrocarbon stream 105. This level of catalyst deactivation compounds can be achieved in the purified aromatic hydrocarbon stream 105 by the reboiler 130 without use of the sacrificial metal alkyl compound. Addition of the sacrificial metal alkyl compound into the process and system 100 upstream of the purified aromatic hydrocarbon stream 105 produces lower concentrations of the catalyst deactivation compounds in the purified aromatic hydrocarbon stream 105.

To control the flow of sacrificial metal alkyl compound to the process and system 100, the process and system 100 can include a control feedback loop. The control feedback loop can include a temperature indicator 131 operably coupled with the reboiler 130 to measure and indicate the temperature of the process liquid (the liquid bottoms received via stream 103 from the distillation column 110) in the reboiler 130. In additional or alternative aspects, the control feedback loop can include the temperature controller 131 operably coupled with stream 105 to measure and indicate the temperature of the process liquid in stream 105. The control feedback loop can also include a flow controller 171 and control valve 170. The temperature indicator 131 is operably connected to the flow controller 171 for the control valve 170 that controls the flow rate of the sacrificial metal alkyl compound into the process and system 100. The control valve 170 is located in a sacrificial metal alkyl compound stream, and the location alternatives for the sacrificial metal alkyl compound stream(s) are explained in more detail below. In aspects, a control valve 170 can be located in any combination of streams that are used to introduce the sacrificial metal alkyl compound to the process and system 100.

The flow controller 171 can be any control equipment or module configured to actuate the control valve to an open position, to a closed position, or to an intermediate position having a 1-99% open status based on the open position having 100% open status and the closed position having 0% open status.

The flow controller 171 is operably connected to a temperature indicator 131. The temperature indicator 131 is i) operably connected to the process liquid side of the reboiler 130 and configured to measure a temperature of the process liquid in the reboiler 130 and communicate (e.g., send a signal representing the temperature) the temperature to the flow controller 171, ii) operably connected to the process liquid in stream 105 and configured to measure a temperature of the process liquid in the stream 105 and communicate (e.g., send a signal representing the temperature) the temperature to the flow controller 171, or iii) operable connected to both the process liquid side of the reboiler 130 and to the process liquid in the stream 105 and configured to measure and communicate the temperature as described. The flow controller 171, in turn, receives the signal and performs a control logic to determine a position for the control valve 170 based on the control logic.

In aspects, the control logic can include comparing the process liquid temperature to a setpoint temperature and determining a position of the control valve 170 based on the comparison. The position of the control valve 170 can be increased to a greater percentage open than a prior position based on determining the temperature of the process liquid (e.g., in the reboiler 130, in stream 105, or both) is below the setpoint (e.g., a boiling point of the aromatic compound). Alternatively, the position of the control valve 170 can be decreased to a lesser percentage open than a prior position based on determining the temperature of the process liquid is at or above the setpoint (e.g., the temperature is close to, at, or above the boiling point of the aromatic compound). In an embodiment, the set point temperature is at or near the boiling point of the aromatic compound at the pressure in the reboiler 130 and/or stream 105. For example, when the aromatic compound is benzene and the reboiler 130 and/or stream 105 operates at atmospheric pressure, the set point temperature is 80.1° C. In aspects where the distillation column 110 operates at pressures higher than atmospheric pressure, the setpoint temperature can be the boiling point of the aromatic compound at corresponding pressure.

In aspects, the sacrificial metal alkyl compound is introduced such that a mole ratio of the sacrificial metal alkyl compound to the catalyst deactivation compounds in the liquid where introduction is made can be in a range of from 1:1 to 1:10, alternatively from 1:1 to 1:6, alternatively, from 1:3 to 1:5, for example.

In some aspects, the concentration of the catalyst deactivation compound(s) in the process liquid can be determined by the control logic using the temperature of the process liquid in the reboiler 130. In these aspects, the control logic can include converting the process liquid temperature to a concentration of catalyst deactivation compounds in the liquid, comparing the concentration to a setpoint concentration, and determining a position of the control valve 170 based on the comparison. In aspects, the conversion of process liquid temperature to a concentration of catalyst deactivation compounds can assume that all catalyst deactivation compounds are water. It was found that for every 1° C. that the temperature of the process liquid is less than the boiling point of aromatic hydrocarbon, there is 15 ppmw (ppm by weight) water present in the process liquid based on the total weight of the process liquid. Based on the 1:1 mole ratio, every 15 ppmw water that is present as determined by the control logic, about 8×10−7 moles of sacrificial metal alkyl compound can be introduced to the process and system 100 in a stream discussed herein, to convert the catalyst deactivation compound(s) to nonreactive compounds. Even though the amount of sacrificial metal alkyl compound is determined based on an assumption that the catalyst deactivation compound is water, it has been found that the amount of the sacrificial metal alkyl compound calculated using this assumption in the control logic is enough to completely convert all of the catalyst deactivation compounds present in the process liquid into nonreactive compounds.

The temperature indicator 131 can send the signal to the flow controller 171 periodically, such as every 0.1 seconds, every 1 second, every 10 seconds, every 1 minute, or every 5 minutes. The flow controller 171 can perform the control logic periodically, such as every time the signal is received from the temperature indicator 131; or alternatively, when the signal from the temperature indicator 131 is continuous, the flow controller 171 can perform the control logic every time the signal changes, to determine the percent open the control valve 170, and adjust the position of the control valve 170 if an adjustment is needed.

The sacrificial metal alkyl compound is introduced to the process and system 100 via stream 108A, stream 108B, stream 108C, stream 108D, stream 108E, stream 108F, or combinations thereof. Streams 108A, 108B, 108C, 108D, 108E, and 108F are illustrated in dashed lines.

In aspects, the sacrificial metal alkyl compound can be introduced as a neat composition (e.g. 100 wt % sacrificial metal alkyl compound), or alternatively, the sacrificial metal alkyl compound can be introduced in a carrier liquid (e.g., a process compatible hydrocarbon such as benzene, cyclohexane, or mixture thereof), where the concentration of the sacrificial metal alkyl compound in in the carrier liquid is 0.01 wt % to 99.9 wt % of the sacrificial metal alkyl compound based on a total weight of the carrier liquid.

In some aspects for the process and system 100 in FIG. 1, the sacrificial metal alkyl compound can be introduced in stream 108A, stream 108B, stream 108C, or combinations thereof. In further aspects, in addition to one or a combination of stream108A, stream 108B, and stream 108C, the sacrificial metal alkyl compound can be introduced in stream 108D, 108E, 108F, or combinations thereof.

Any stream 108A, 108B, 108C, 108D, 108E, or 108F that is utilized in process and system 100 contains a respective control valve that is the same as control valve 170 and connected to the flow controller 171, which receives the signal indicative of the process liquid temperature in the reboiler 130, as discussed above.

In aspects where the sacrificial metal alkyl compound is introduced by stream 108A, stream 108A is connected to process liquid side of the reboiler 130 (the shell side of the reboiler 130 that receives the bottoms stream 103), and configured to introduce the sacrificial metal alkyl compound into the process liquid inside the reboiler 130 (e.g., on the shell side of the reboiler 130).

In aspects where the sacrificial metal alkyl compound is introduced by stream 108B, stream 108B is connected to the recycle stream 104. Stream 108B can contain a control valve that is the same as control valve 170 and connected to the flow controller 171, which receives the signal indicative of the process liquid temperature in the reboiler 130, as discussed above.

In aspects where the sacrificial metal alkyl compound is introduced by stream 108C, stream 108C is connected to the purified aromatic hydrocarbon stream 105. Stream 108C can contain a control valve that is the same as control valve 170 and connected to the flow controller 171, which receives the signal indicative of the process liquid temperature in the reboiler 130, as discussed above. The location where stream 108C is connected to the purified aromatic hydrocarbon stream 105 is upstream of any hydrogen introduction (e.g., via stream 121B to the purified aromatic hydrocarbon stream 105.

In aspects where the sacrificial metal alkyl compound is introduced by stream 108D, stream 108D is connected to a hydrogen feed stream 121B, which in turn, combines with the purified aromatic hydrocarbon stream 105 to form combined stream 106. Stream 108D can contain a control valve that is the same as control valve 170 and connected to the flow controller 171, which receives the signal indicative of the process liquid temperature in the reboiler 130, as discussed above.

In aspects where the sacrificial metal alkyl compound is introduced by stream 108E, stream 108E is connected to the combined stream 106 that is formed by combining the hydrogen feed stream 121B and the purified aromatic hydrocarbon stream 105. Stream 108E can contain a control valve that is the same as control valve 170 and connected to the flow controller 171, which receives the signal indicative of the process liquid temperature in the reboiler 130, as discussed above. The location where stream 108E is connected to the combined stream 106 is downstream of hydrogen introduction (e.g., via stream 121B) and upstream of the hydrogenation reactor 120.

In aspects where the sacrificial metal alkyl compound is introduced by stream 108F, stream 108F is connected to the hydrogen feed stream 121A that is connected directly to the hydrogenation reactor 120. Stream 108F can contain a control valve that is the same as control valve 170 and connected to the flow controller 171, which receives the signal indicative of the process liquid temperature in the reboiler 130, as discussed above. The location where stream 108F is connected to hydrogen feed stream 121A is upstream of the liquid phase hydrogenation reactor 120. Stream 108F can be used as a supplemental source of sacrificial metal alkyl compound, that is supplemental to the sacrificial metal alkyl compound introduced by one or more of stream 108A, stream 108B, stream 108C, stream 108D, or stream 108E, at least because the sacrificial metal alkyl compound introduced by stream 108F does not convert the catalyst deactivation compounds to nonreactive products upstream of the liquid phase hydrogenation reactor 120 (since the sacrificial metal alkyl compound would convert such compounds inside the liquid phase hydrogenation reactor 120).

In the process and system of FIG. 1, the sacrificial metal alkyl compound that is introduced by stream 108A, stream 108B, stream 108C, stream 108D, stream 108E, stream 108F, or combinations thereof, converts catalyst deactivation compounds to nonreactive compounds that flow in purified aromatic hydrocarbon stream 105 or combined stream 106 to the liquid phase hydrogenation reactor 120. Thus, in the process and system of FIG. 1, the nonreactive solid products contained in the nonreactive compounds are not removed from the process and system 100 upstream of the liquid phase hydrogenation reactor 120. Instead, the nonreactive solid products can be removed along with solid spent catalyst from the hydrogenation reactor 120, for example via solids removal stream 128.

The process and system 100 can include a liquid phase hydrogenation reactor 120, and a liquid cooling loop 140 connected to a liquid outlet on the bottom of the liquid phase hydrogenation reactor 120 and to a liquid inlet on a side of the liquid phase hydrogenation reactor 120.

The purified aromatic hydrocarbon stream 105 or the combined stream 106 (depending on the embodiment) can be connected to an inlet on the side of the liquid phase hydrogenation reactor 120, and thus, can be a hydrogenation reactor feed stream. In embodiments that include hydrogen feed stream 121A, the hydrogen feed stream 121A can be fluidly coupled to the inlet for stream 105 or 106 or a second inlet on the liquid phase hydrogenation reactor 120.

The aromatic hydrocarbon is introduced to the liquid phase hydrogenation reactor 120 via the purified aromatic hydrocarbon stream 105 or the combined stream 106, hydrogen can be introduced to the liquid phase hydrogenation reactor 120 via the hydrogen feed stream 121A (and/or via the combined stream 106 when hydrogen feed stream 121B combines with purified aromatic hydrocarbon stream 105), and cooled reaction medium can be introduced to the liquid phase hydrogenation reactor 120 via the liquid cooling loop 140.

In various aspects, a homogeneous catalyst system can be introduced to the liquid phase hydrogenation reactor 120 via the purified aromatic hydrocarbon stream 105, via the hydrogen feed stream 121A, via the combined stream 106, via the recycle stream 126, or via another via a catalyst stream that is fluidly connected to the liquid phase hydrogenation reactor 120.

In FIG. 1, the catalyst stream 127 is connected to stream 125 in the liquid cooling loop 140 to form recycle stream 126, and the homogeneous catalyst can flow into the liquid phase hydrogenation reactor 120 via stream 126. The homogeneous catalyst can be added to the recycle stream 126 continuously or periodically, and the catalyst injection rate can vary depending on the application, for example between about 0.45 kg/hr to about 226.8 kg/hr (1 lb/hr to about 500 lb/hr).

The purified aromatic hydrocarbon stream 105 or combined stream 106 can include any feed hydrocarbon having a carbon-carbon double bond. In aspects, the hydrocarbon can include a linear olefin, a branched olefin, a cyclic olefin, or combinations thereof. In more particular aspects, the feed hydrocarbon is one or more aromatic hydrocarbons such as benzene, toluene, a xylene, styrene, or combinations thereof. The purified aromatic hydrocarbon stream 105 or combined stream 106 can be fed to the liquid phase hydrogenation reactor 120 as a liquid phase. In some aspects, the purified aromatic hydrocarbon stream 105 or combined stream 106 can include cyclohexane.

Hydrogen feed stream 121A or 121B can be a gas containing hydrogen. In aspects, the hydrogen feed stream 121A or 121B can include hydrogen in an amount of greater than or equal to 90, 91, 92, 93, 94, 95, 96, 97, 98, 99, or 100 vol % based on a total volume of the hydrogen feed stream 121A or 121B. In some aspects, the hydrogen feed stream 121A or 121B can include hydrogen, methane, ethane, propane, n-butane, isobutene, water, or combinations thereof. The flow rate of gas in hydrogen feed stream 121A or 121B can be such that hydrogen is present in the liquid phase hydrogenation reactor 120 in excess of the stoichiometric amount needed to hydrogenate the feed hydrocarbon.

Liquid reactor effluent stream 123 can include a portion of the liquid reaction medium that is withdrawn from the liquid phase hydrogenation reactor 120. The liquid reaction medium can contain hydrogenation product, unreacted feed hydrocarbon(s), homogeneous catalyst that has not converted to solid particulate, solid particulates (e.g., comprising catalyst decomposition product), or combinations thereof. In the process and system 100 of FIG. 1, the solid particulates can also include the nonreactive solid products formed upstream of the liquid phase hydrogenation reactor 120.

In aspects, liquid reactor effluent stream 123 can include the hydrogenation product in an amount of from about 20 vol % to about 99 vol %; alternatively, from about 50 vol % to about 99 vol %; alternatively, from about 70 vol % to about 99 vol % based on a total volume of the liquid reactor effluent stream 123.

The gas phase product stream 122 can include the hydrogenation product (e.g., cyclohexane), unreacted hydrocarbon feed (e.g., benzene), unreacted hydrogen, or combinations thereof. The gas phase product stream 122 can include hydrogenation product (e.g., cyclohexane) in a range of from about 50 vol % to about 99.8 vol % based on a total volume of the gas phase product stream 122. In additional aspects, the gas phase product stream 122 can include unreacted hydrogen in a range of from about 20 vol % to about 40 vol % based on a total volume of the gas phase product stream 122.

The heat exchanger 160 can be embodied as a shell and tube heat exchanger, a thermosyphon-type heat exchanger, a jacketed heat exchanger, or a vertical calendria-type evaporator. When cooling medium is utilized such as in a shell and tube heat exchanger, coolant stream 161 can be utilized. The coolant stream 161 can include any coolant, and can be embodied as cooling water. The warmed coolant stream 162 can include a warmed coolant. In aspects, the warmed coolant stream contains warmed coolant in the form of steam. In aspects, the steam in warmed coolant stream 162 can have a pressure in a range of from about 482 kPag (70 psig) to about 1103 kPag (160 psig); alternatively, from about 482 kPag) (70 psig) to about 965 kPag (140 psig); alternatively, from about 482 kPag (70 psig) to about 861 kPag (125 psig). In aspects, the steam in warmed coolant stream 162 can have a temperature of less than or equal to about 204° C. (400° F.).

The homogeneous catalyst can include a catalyst operable to catalyze the liquid phase hydrogenation of a hydrocarbon disclosed herein to form a hydrogenation product as disclosed herein (e.g., aromatic compound to produce a cycloalkane compound; e.g., benzene to produce cyclohexane). In aspects, the catalyst comprises a metal from an IUPAC Group 10 of the periodic table such as nickel, platinum, palladium, iron, or a combination thereof. The Group 10 metal can be contained in a Group 10 metal compound that is formed by using an aluminum alkyl compound to reduce a liquid carrier containing a nickel carboxylate, a sodium carboxylate, or a combination thereof, to form the homogeneous catalyst. Any nickel carboxylate and sodium carboxylate can generally be a derivatives of an aliphatic carboxylic acid. The nickel carboxylate can have the formula R—COONi, where R is a linear or branched alkyl group having containing 1 to 24 carbon atoms. The sodium carboxylate can have the formula R—COONa, where R is a linear or alkyl group having 1 to 24 carbon atoms. When the nickel carboxylate and sodium carboxylate compounds are used in combination, a Ni:Na molar ratio can be in a range of from 2:1 to 1000:1.

In aspects, the catalyst is soluble or colloidal in the liquid carrier (e.g., a saturated hydrocarbon), to form the homogeneous catalyst of this disclosure. In aspects, the catalyst remains in solution or in suspension in the liquid reaction medium disclosed herein, after introduction into the liquid phase hydrogenation reactor 120 with the liquid carrier and prior to reaction with the aromatic compound. For example, the homogeneous catalyst can be formed by adding a trialkyl aluminum compound to a nickel carboxylate and a sodium carboxylate dissolved or suspended in a hydrocarbon or mixture of hydrocarbons, as described, for example, in U.S. Pat. No. 5,668,293, the disclosure of which is hereby incorporated herein.

In aspects where the homogeneous catalyst is formed by mixing one or more IUPAC Group 10 metal compounds and one or more aluminum alkyl compounds, the one or more aluminum alkyl compounds can be introduced to the reactor 120 together or separately from the one or more Group 10 metal compounds (e.g., together in stream 127 or separately in different streams that are each fluidly coupled to the liquid phase hydrogenation reactor 120). The aluminum alkyl compounds can be introduced neat or in a liquid carrier such as cyclohexane or benzene. The Group 10 metal compound(s) can be introduced in a liquid carrier as well. In such cases, the one or more Group 10 metal compounds can be introduced continuously and the one or more aluminum alkyl compounds can also be introduced continuously. Alternatively, the one or more Group 10 metal compounds can be introduced intermittently and the one or more aluminum alkyl compounds can be introduced intermittently such that the one or more aluminum alkyl compounds is introduced between the periods of time when the one or more Group 10 metal compounds are introduced. Continuous or intermittent introduction of the aluminum alkyl compound(s) in an amount greater than is stoichiometrically required for forming the homogeneous catalyst can improve the reactivity of the one or more Group 10 metal compounds with the aromatic compound because the excess amount of aluminum alkyl compound can be an amount of sacrificial metal alkyl compound that reacts with any catalyst deactivation compounds that are in liquid reaction medium in the liquid phase hydrogenation reactor 120. Introducing the alkyl aluminum compound into the liquid phase hydrogenation reactor 120 during a period when the Group 10 metal compound(s) is/are not introduced into the liquid phase hydrogenation reactor 120 can convert any new catalyst deactivation compounds that are fed to the liquid phase hydrogenation reactor 120 to the nonreactive compounds (e.g., in some cases, including nonreactive solid products), avoiding reaction of the new catalyst deactivation compounds with the Group 10 metal compound(s) when catalyst flow resumes (e.g., in the embodiments with intermittent introduction of the Group 10 metal compound(s)).

The liquid cooling loop 140 is configured to cool the liquid reaction medium that is withdrawn from the liquid phase hydrogenation reactor 120, and to return the cooled liquid reaction medium to the liquid phase hydrogenation reactor 120. The liquid cooling loop 140 can include a liquid reactor effluent stream 123, a pump 150, a pumped stream 124, a heat exchanger 160, a cooled stream 125, a recycle stream 126, a coolant stream 161, and a warmed coolant stream 162.

The liquid reactor effluent stream 123 containing withdrawn liquid reaction medium can be connected to the liquid phase hydrogenation reactor 120 and to an inlet of the pump 150. The pumped stream 124 containing the withdrawn liquid reaction medium can be connected to the outlet of the pump 150 and to an inlet of the heat exchanger 160. In the heat exchanger 160, the liquid reaction medium is cooled by heat exchange contact with a cooling medium. In aspects, the reduction in temperature of the liquid reaction medium is in a range of 2.7° C. to 11.1° C. (5° F. to 20° F.), for example, reduced by about 5.5° C. (10° F.). The cooled stream 125 containing cooled liquid reaction medium can be connected to the outlet of the heat exchanger 160 and to the recycle stream 126. The catalyst stream 127 can also be connected to the recycle stream 126. The recycle stream 126 can be connected to the cooling loop inlet of the liquid phase hydrogenation reactor 120.

The liquid phase hydrogenation reactor 120 can contain a liquid reaction medium comprising the unreacted aromatic hydrocarbon, hydrogenation product, hydrogen, a homogeneous catalyst system, solid particulates (including spent catalyst), or combinations thereof. The liquid phase hydrogenation reactor 120 is configured to hydrogenate the feed hydrocarbon (e.g., an aromatic hydrocarbon such as benzene) with hydrogen in a presence of the homogeneous catalyst system to form a gas phase product stream 122 comprising a hydrogenation product (e.g., a saturated hydrocarbon such as cyclohexane). In aspects, the temperature of the liquid reaction medium in the liquid phase hydrogenation reactor 120 can be in a range of from about 125° C. to about 275° C.; alternatively, from about 170° C. to about 230° C.; alternatively, from about 100° C. to about 200° C.; alternatively, from about 100° C. to about 250° C. In some aspects, the temperature is below a cracking temperature for olefins that may be present in the liquid reaction medium and received from the purified aromatic hydrocarbon stream 105 or combined stream 106 (e.g., a temperature less than about 250° C.), to prevent hydrocracking of a hydrocarbon feed. In aspects, the pressure of the liquid reaction medium in the liquid phase hydrogenation reactor 120 can be sufficient to maintain a liquid phase in the liquid phase hydrogenation reactor 120, and may be in the range of from 0.5 MPa to 10 MPa (about 5 atm to about 100 atm); alternatively, from 2 MPa to 3 MPa (about 20 atm to about 30 atm); alternatively, from 1.5 MPa to 3 MPa (about 15 atm to about 30 atm).

Hydrogenation reaction takes place in the liquid phase hydrogenation reactor 120 such that the hydrogenation product in gas phase flows from the liquid phase hydrogenation reactor 120 in gas phase product stream 122 and liquid reaction medium flows from the liquid phase hydrogenation reactor 120 via liquid reactor effluent stream 123.

Liquid phase effluent can be withdrawn from liquid phase hydrogenation reactor 120 in liquid reactor effluent stream 123 to the pump 150. Pump 150 is configured to withdraw a portion of the liquid reaction medium from the liquid phase hydrogenation reactor 120 and to pump the withdrawn liquid reaction medium through the heat exchanger 160 and back into the liquid phase hydrogenation reactor 120 via the recycle stream 126. The temperature of the withdrawn liquid reaction medium is reduced in the heat exchanger 160 via heat exchange with a heat exchange medium (e.g., coolant such as cooling water). In aspects, the heat exchanger 160 is configured to reduce the temperature of the withdrawn liquid reaction medium by 2.7° C. to 11.1° C. (about 5° F. to about 20° F.). Cooled reaction medium can flow from the heat exchanger 160 into the recycle stream 126, for return to the liquid phase hydrogenation reactor 120.

The solid particulates (e.g., including the nonreactive solid products formed upstream of the liquid phase hydrogenation reactor 120 and spent catalyst solids formed in the liquid phase hydrogenation reactor 120) can be removed from the process and system 100 via stream 128. A control valve is illustrated in stream 128 as an example of equipment that can be used to remove the solid particulates from the process and system 100.

The above description can apply to steady state operation of the process and system 100. On startup, the presence of catalyst deactivation compounds in the equipment of the process and system 100 can be a challenge, in that, these compounds should be minimally present as possible so as to not poison the hydrogenation catalyst on startup. In startup, initial contents for the distillation column 110 and the liquid reaction medium in the hydrogenation reactor 120 can be added, such as cyclohexane. The remainder of the process and system 100 can then be filled and purged with nitrogen gas for an amount of time that is called the drying time. The flow equipment such as any pump in the purified aromatic hydrocarbon stream 105 and pump 150 in the liquid cooling loop 140 can be started, and the heat exchangers (e.g., condenser 115, reboiler 130, heat exchanger 160) can be turned on. Hydrogenation catalyst flow into the liquid phase hydrogenation reactor 120 is then initiated, and feed stream 101 flow is initiated, not necessarily in that order. At any point in the above startup steps, the sacrificial metal alkyl compound can be introduced at one or more locations described above (e.g., stream 108A, stream 108B, stream 108C, stream 108D, stream 108E, stream 108F, or combinations thereof), so that the sacrificial metal alkyl compound reacts with any catalyst deactivation compounds in the process and system 100.

In aspects of startup, the sacrificial metal alkyl compound can be introduced as a large dose, for example, an amount in a range of from 22.6 kg to 45.4 kg (50 lb to 100 lb). Alternatively in startup, introduction of the sacrificial metal alkyl compound can be started at a low level and increased over time to a flow rate which can be used at steady state operation of the process and system 100. Introduction of the sacrificial metal alkyl compound on startup and before nitrogen purge can reduce the drying time because the sacrificial metal alkyl compound can convert at least part of the water contained in the process and system 100 on startup (water being one of the catalyst deactivation compounds described herein) to nonreactive compounds.

The process and system 100 of FIG. 1 can increase hydrogenation catalyst life (longer useful life) even though the nonreactive solid products flow into liquid phase hydrogenation reactor 120 because the catalyst deactivation compound(s) are converted to nonreactive compounds that do not react with the hydrogenation catalyst in the liquid phase hydrogenation reactor 120. Increasing the catalyst life increases the run time between shutdowns and turnaround of the process and system 100, which overall increases the percentage of time that the process and system 100 is online compared to a similar process and system that does not introduce the sacrificial metal alkyl compound as described herein.

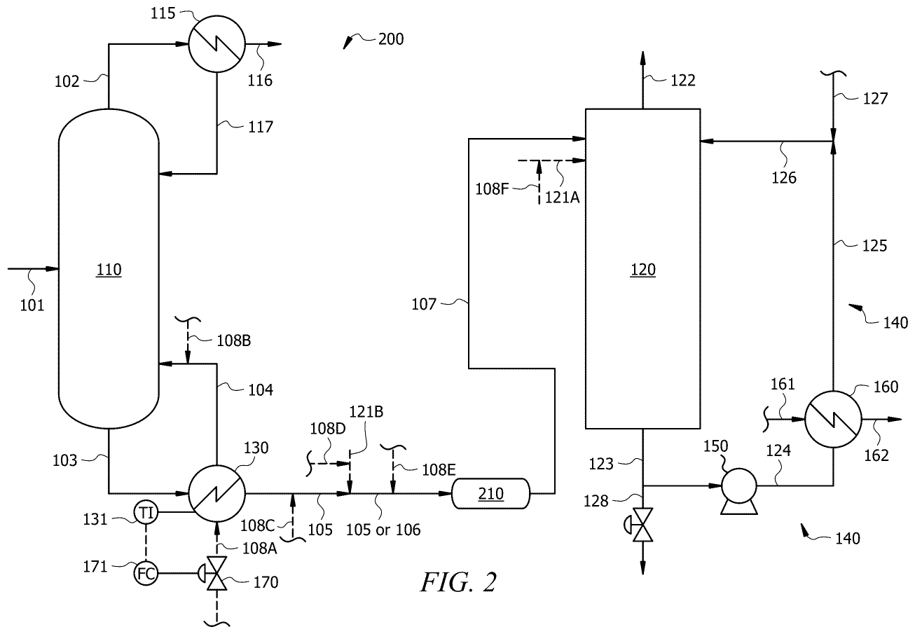

FIG. 2 illustrates a schematic diagram of another process and system 200 for converting hydrogenation catalyst deactivation compounds to nonreactive compounds upstream of a liquid phase hydrogenation reactor 120, where nonreactive solid products are removed upstream of the liquid phase hydrogenation reactor 120 by a separator 210.

The aspects, embodiments, equipment, functionality, and processing of the process and system 200 is similar to that described for the process and system 100, and similar description is not reproduced here. The process and system 200 in FIG. 2 differs from the process and system 100 in FIG. 1, in that, a separator 210 is included upstream of the liquid phase hydrogenation reactor 120 to remove the nonreactive solid products from the liquid aromatic hydrocarbon prior to the liquid aromatic hydrocarbon being introduced to the liquid phase hydrogenation reactor 120.

The nonreactive solid products can precipitate or settle out of solution and deposit on various interior surfaces of the equipment, such as piping and valves, the liquid phase hydrogenation reactor 120, internals of the liquid phase hydrogenation reactor 120, the pump(s), the heat exchanger(s), and the like. Over time, the nonreactive solid products can accumulate as deposits on the interior surfaces, and the deposits can cause problems including but not limited to restriction of flow (fouling) and mal distribution of the liquid in the equipment and corrosion of the equipment materials. Thus, removal of the nonreactive solid products upstream of the liquid phase hydrogenation reactor 120 prevents deposit accumulation in the liquid phase hydrogenation reactor 120 and in the liquid cooling loop 140 and associated equipment in the liquid cooling loop 140.

As illustrated in FIG. 2, the nonreactive solid products are removed upstream of the liquid phase hydrogenation reactor 120 using the separator 210. The separator 210 can be embodied as a static mixer, an adsorbent bed, a hydrocyclone, or combinations thereof connected in series and/or in parallel in in any order. In FIG. 2, the entire amount of the stream 105 or 106 can be introduced to the separator 210. In alternative embodiments, a portion of the stream 105 or 106 can be introduced to the separator 210 while another portion bypasses the separator 210. The bypassed portion can combine with the portion that flows through separator 210 to form stream 107, prior to being introduced as stream 107 to the liquid phase hydrogenation reactor 120.

To move the aromatic hydrocarbon into the separator 210, the purified aromatic hydrocarbon stream 105 can include motive equipment, such as one or more pumps to move the aromatic compound downstream. In some aspects, the motive equipment, e.g., pump, can generate a pumped stream that feeds to the separator 210. In aspects, a pressure of the pumped stream is higher than a pressure of the purified aromatic hydrocarbons stream 105, and the pressure is sufficient to move the aromatic hydrocarbon liquid through the separator 210 and to the liquid phase hydrogenation reactor 120.

The separator 210 has an inlet connected to the purified aromatic hydrocarbon liquid stream 105 and an outlet connected to the hydrogenation reactor feed stream 107. In aspects where the separator 210 is embodied as a vessel that moves nonreactive solid products out of the vessel while in operation, a solid stream can be connected to a second outlet (for solids) of the separator 210.

In embodiments where the separator 210 includes a static mixer, the static mixer can be a vessel (e.g., a section of larger sized pipe) fitted with internal baffles to disrupt flow therethrough. The internal surfaces of the vessel and baffles can intentionally cause accumulation and deposit of the nonreactive solid products. The static mixer serves as a sacrificial equipment for collection of the nonreactive solid products on the internal surfaces thereof.

In embodiments where the separator 210 includes an adsorbent bed, the adsorbent bed can include an adsorbent material such as activated carbon, clay, alumina, silica, or the like, and remaining trace impurities can adsorb onto the adsorbent material of the adsorbent bed. The adsorbent can adsorb, in some embodiments, some of the nonreactive compounds that are not the nonreactive solid products. In aspects, the adsorbent can also function as a filter by trapping at least some of the nonreactive solids therein without adsorbing the nonreactive solids. The adsorbent bed can be embodied as a vessel containing packing or adsorbent, or any other device with internal surface area sufficient to collect the nonreactive solid products.

In embodiments where the separator 210 includes a hydrocyclone, the hydrocyclone can be a conically shaped vessel that receives the slurry (liquid and solids) of aromatic hydrocarbon and nonreactive solid products from the purified aromatic hydrocarbon stream 105 of the combined stream 106 and separates, through cyclonic separation action, the stream 105 or stream 106 into a stream containing the nonreactive solid products and a hydrogenation reactor feed (e.g., in stream 107) containing the aromatic hydrocarbon. In operation, due to centrifugal forces the heavier nonreactive solid products flow radially outward further than the liquid, spiraling in a downward flow direction to a bottom outlet of the hydrocyclone, while the comparatively lighter liquid molecules change direction upward when the liquid molecules approach the cone apex and flow through the vortex finder of the hydrocyclone, and out of a top outlet of the hydrocyclone.

The separator 210 produces the hydrogenation reactor feed stream 107, which is connected to the separator 210 and to the liquid phase hydrogenation reactor 120. Stream 107 can contain the aromatic hydrocarbon in liquid phase in an amount of greater than 95, 96, 97, 98, 99, 99.9, 99.99 wt %, and contains less than 1, 0.1, 0.01, or 0.001 wt % of nonreactive solid products based on a total weight of the stream 107.

The process and system 200 of FIG. 2 can increase hydrogenation catalyst life (longer useful life) because the catalyst deactivation compound(s) are converted to nonreactive compounds that do not react with the hydrogenation catalyst in the liquid phase hydrogenation reactor 120. Increasing the catalyst life increases the run time between shutdowns and turnaround of the process and system 200, which overall increases the percentage of time that the process and system 200 is online compared to a similar process and system that does not introduce the sacrificial metal alkyl compound as described herein. Further, the process and system 200 can reduce fouling due to any accumulation of deposits of the nonreactive solid products in the liquid phase hydrogenation reactor 120 or liquid cooling loop 140, because the nonreactive solid products are removed by the separator 210 and are not contained in a stream that feeds to the liquid phase hydrogenation reactor 120.

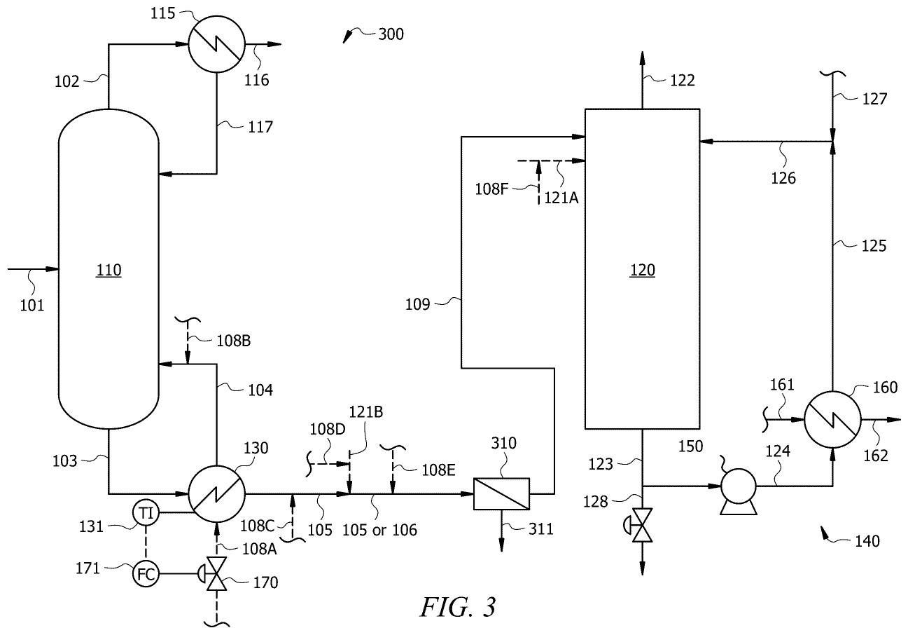

### FIG. 3

FIG. 3 illustrates a schematic diagram of another process and system 300 for converting hydrogenation catalyst deactivation compounds to nonreactive compounds upstream of a liquid phase hydrogenation reactor 120, where nonreactive solid products are removed upstream of the liquid phase hydrogenation reactor 120 by a filter 310.

The aspects, embodiments, equipment, functionality, and processing of the process and system 300 is similar to that described for the process and system 100, and similar description is not reproduced here. The process and system 300 in FIG. 3 differs from the process and system 100 in FIG. 1, in that, a filter 310 is included upstream of the liquid phase hydrogenation reactor 120 to remove the nonreactive solid products from the liquid aromatic hydrocarbon prior to the liquid aromatic hydrocarbon being introduced to the liquid phase hydrogenation reactor 120.

It is to be understood that the filter 310 can be utilized in combination with the separator 210, upstream or downstream of the separator 210. In FIG. 3, the entire amount of the stream 105 or 106 can be introduced to the filter 310. In alternative embodiments, a portion of the stream 105 or 106 can be introduced to the filter 310 while another portion bypasses the filter 310. The bypassed portion can combine with the permeate of the filter 310 to form stream 109, prior to being introduced as stream 109 to the liquid phase hydrogenation reactor 120.

The filter 310 can be embodied as a housing having one or more filter elements contained therein. Each filter element of the filter 310 is configured to separate molecules and particles based on the size of the molecules and particles relative to a pore size of pores in the filter element. Molecules and particles smaller than the pore size of the filter element pass through the filter element as a permeate to a permeate side of the filter 310, and molecules and particles larger than the pore size of the filter element are retained as retentate on a retentate side of the filter 310.

In aspects, the pores of the filter element have a pore size (e.g., an average pore size) in a range of from about 1 micron or to about 100 microns; alternatively, 1 to 30 microns; alternatively, 1 to 25 microns; alternatively, 1 to 20 microns; alternatively, 10 to 30 microns; alternatively, 10 to 25 microns; alternatively, 10 to 20 microns; alternatively, 20 to 25 microns.

In aspects, the filter element of the filter 310 can be made of a ceramic material, a metal (e.g., stainless steel) material, a polymeric material, or combinations thereof. In several aspects, the filter element can be a polymeric material that includes cellulose acetate, cellulose nitrate, polyamide, polycarbonate, polyethersulphone, polytetrafluoroethylene, or combinations thereof. In aspects, the filter 310 can be a membrane filter.

Commercial examples of the filter 310 can include a clarifying filter, such as a rotary drum filter or a pressure leaf filter. In commercial operation, a filter cake can form on the retentate side of the filter 310, and there may be a need to break up and/or remove the filter cake (or at least portions of the filter cake). The filter 310 can have a mechanism for “blowback” of the filter cake, such as a pressurized stream of inert hydrocarbon that flows from the permeate side of the filter 310 to the retentate side of the filter 310, dislodging and breaking up portions of the filter cake for removal via the retentate stream 311. Blowback can be initiated upon a differential pressure between the permeate side and the retentate side reaching a hydraulic threshold.

The filter 310 can have a permeate outlet connected to the permeate stream 109 and a retentate outlet connected to the retentate stream 311. The permeate stream 109 is configured to allow permeate to flow from the filter 310 to the hydrogenation reactor 120, and the retentate stream 311 is configured to allow retentate to flow from the filter 310 for storage, disposing, or further processing. In aspects, the permeate in the permeate stream 109 includes the aromatic hydrocarbon and is the hydrogenation reactor feed stream. The permeate can also include nonreactive compounds that are not solids. In aspects, the retentate in the retentate stream 311 can include the nonreactive solid products.

In aspects, the filter 310 removes from about 95 wt % to about 99 wt %; alternatively, from about 96 wt % to about 99 wt %; alternatively, from about 97 wt % to about 99 wt %; alternatively, from about 98 wt % to about 99 wt % of the nonreactive solid products that are contained in the purified aromatic hydrocarbon stream 105 or combined stream 106 that is received by the filter 310 (based on a total weight of the stream 105 or stream 106). Stream 109 can contain the aromatic hydrocarbon in liquid phase in an amount of greater than 95, 96, 97, 98, 99, 99.9, 99.99 wt %, and contains less than 5, 4, 3, 2, or 1 ppmw of nonreactive solid products based on a total weight of the stream 109.

The permeate stream 109 connects to the liquid phase hydrogenation reactor 120 as the hydrogenation reactor feed stream and is configured to flow the aromatic compound having reduced-solids content compared to the purified aromatic hydrocarbon stream 105 or combined stream 106 to the liquid phase hydrogenation reactor 120.

The process and system 300 of FIG. 3 can increase hydrogenation catalyst life (longer useful life) because the catalyst deactivation compound(s) are converted to nonreactive compounds that do not react with the hydrogenation catalyst in the liquid phase hydrogenation reactor 120. Increasing the catalyst life increases the run time between shutdowns and turnaround of the process and system 300, which overall increases the percentage of time that the process and system 300 is online compared to a similar process and system that does not introduce the sacrificial metal alkyl compound as described herein. Further, the process and system 300 can reduce fouling due to any accumulation of deposits of the nonreactive solid products in the liquid phase hydrogenation reactor 120 or liquid cooling loop 140, because the nonreactive solid products are removed by the filter 310 and are not contained in a stream that feeds to the liquid phase hydrogenation reactor 120.

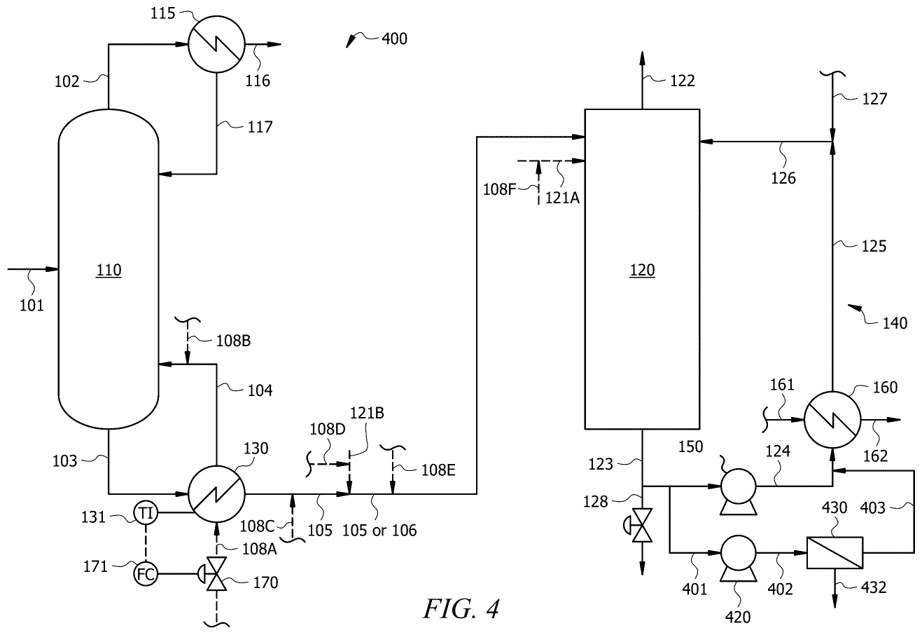

FIG. 4 illustrates a schematic diagram of another process and system 400 for converting hydrogenation catalyst deactivation compounds to nonreactive compounds upstream of a liquid phase hydrogenation reactor 120, where nonreactive solid products are removed by a filter 430 connected to the liquid cooling loop 140 of the liquid phase hydrogenation reactor 120.

It is to be understood that the filter 430 can be utilized in process and system 200 and in process and system 300.

The aspects, embodiments, equipment, functionality, and processing of the process and system 400 is similar to that described for the process and system 100, and similar description is not reproduced here. The process and system 400 in FIG. 4 differs from the process and system 100 in FIG. 1, in that, a filter 430 is included in the liquid cooling loop 140 associated with the liquid phase hydrogenation reactor 120, to remove the nonreactive solid products in a withdrawn portion of the liquid reaction medium when the withdrawn portion flows in the liquid cooling loop 140, prior to reintroduction of the withdrawn portion of the liquid reaction medium to the liquid phase hydrogenation reactor 120 via the liquid cooling loop 140.

A slip stream 401 can be connected to the stream 123 of the liquid cooling loop 140. The slip stream 401 is configured to receive a portion of the stream 123, e.g., 1 wt % to 10 wt % of the stream 123 based on a total weight of the stream 123. The slip stream 401 is connected to a pump 420, configured to pressurize the liquid to a filtration pressure in stream 402. Stream 402 is connected to an inlet of the filter 430.

The filter 430 can be embodied as a housing having one or more filter elements contained therein. Each filter element of the filter 430 is configured to separate molecules and particles based on the size of the molecules and particles relative to a pore size of pores in the filter element. Molecules and particles smaller than the pore size of the filter element pass through the filter element as a permeate to a permeate side of the filter 430, and molecules and particles larger than the pore size of the filter element are retained as retentate on a retentate side of the filter 430.

In aspects, the pores of the filter element have a pore size (e.g., an average pore size) in a range of from about 1 micron or to about 100 microns; alternatively, 1 to 30 microns; alternatively, 1 to 25 microns; alternatively, 1 to 20 microns; alternatively, 10 to 30 microns; alternatively, 10 to 25 microns; alternatively, 10 to 20 microns; alternatively, 20 to 25 microns.

In aspects, the filter element of the filter 430 can be made of a ceramic material, a metal (e.g., stainless steel) material, a polymeric material, or combinations thereof. In several aspects, the filter element can be a polymeric material that includes cellulose acetate, cellulose nitrate, polyamide, polycarbonate, polyethersulphone, polytetrafluoroethylene, or combinations thereof. In aspects, the filter 430 can be a membrane filter.

Commercial examples of the filter 430 can include a clarifying filter, such as a rotary drum filter or a pressure leaf filter. In commercial operation, a filter cake can form on the retentate side of the filter 430, and there may be a need to break up and/or remove the filter cake (or at least portions of the filter cake). The filter 430 can have a mechanism for “blowback” of the filter cake, such as a pressurized stream of inert hydrocarbon that flows from the permeate side of the filter 430 to the retentate side of the filter 430, dislodging and breaking up portions of the filter cake for removal via the retentate stream 432. Blowback can be initiated upon a differential pressure between the permeate side and the retentate side reaching a hydraulic threshold.

The filter 430 can have a permeate outlet connected to the permeate stream 403 and a retentate outlet connected to the retentate stream 432. The permeate stream 403 is configured to allow permeate to flow from the filter 430, and the retentate stream 432 is configured to allow retentate to flow from the filter 430. In aspects, the permeate in the permeate stream 403 includes the hydrogenation product (e.g., cycloalkane such as cyclohexane and unreacted aromatic hydrocarbon such as benzene). The permeate stream 403 can also include the nonreactive compounds that are not solids. In aspects, the retentate in the retentate stream 432 can include the nonreactive solid products and spent catalyst solids.

In aspects, the filter 430 removes from about 95 wt % to about 99 wt %; alternatively, from about 96 wt % to about 99 wt %; alternatively, from about 97 wt % to about 99 wt %; alternatively, from about 98 wt % to about 99 wt % of the nonreactive solid products that are contained in the stream 402 that is received by the filter 430 (based on a total weight of the stream 402), such that the permeate stream 403 has less than 20, 19, 18, 17, 16, 15, 14, 13, 12, 11, 10, 9, 8, 7, 6, 5, 4, 3, 2, or 1 ppmw nonreactive solid products based on a total weight of the permeate stream 403.

The permeate stream 403 connects to the liquid cooling loop 140 (e.g., to the stream 124) and is configured to flow the liquid reaction medium having reduced-solids content compared to the streams 123, 401, and 402.

The process and system 400 of FIG. 4 can increase hydrogenation catalyst life (longer useful life) even though the nonreactive compounds flow into liquid phase hydrogenation reactor 120 because the catalyst deactivation compound(s) are converted to nonreactive compounds that do not react with the hydrogenation catalyst in the liquid phase hydrogenation reactor 120. Increasing the catalyst life increases the run time between shutdowns and turnaround of the process and system 400, which overall increases the percentage of time that the process and system 400 is online compared to a similar process and system that does not introduce the sacrificial metal alkyl compound as described herein. Further, the process and system 400 can reduce fouling due to accumulation of deposits of the nonreactive solid products in the liquid phase hydrogenation reactor 120 or liquid cooling loop 140, because the nonreactive solid products are removed by the filter 430 to help prevent buildup of the nonreactive solid products in the liquid phase hydrogenation reactor 120 and in the liquid cooling loop 140.

Processes to be implemented in one or more of the discloses processes and systems 100, 200, 300, and 400, include introducing a sacrificial metal alkyl compound to a stream or equipment comprising an aromatic compound and a catalyst deactivation compound, wherein a location of the stream or equipment is upstream of a liquid phase hydrogenation reactor (e.g., via stream 108A, 108B, 108C, 108D, 108E, 108F, or combinations thereof), wherein the location is not in a liquid cooling loop of the liquid phase hydrogenation reactor 120; and reacting the sacrificial metal alkyl compound with the catalyst deactivation compound in the stream or equipment to form a nonreactive compounds containing nonreactive solid products. The process can also include, prior to introducing the sacrificial metal alkyl compound, distilling in the distillation column 110 a crude aromatic mixture comprising the aromatic compound, the catalyst deactivation compound, and water into an overhead product comprising the water and a bottoms product comprising the aromatic compound and the catalyst deactivation compound; and reboiling, in the reboiler 130, the bottoms product to form a recycle stream 104 and a purified aromatic hydrocarbon stream 105 comprising the aromatic compound, wherein aromatic compound is introduced to the liquid phase hydrogenation reactor 120 via the purified aromatic hydrocarbon stream 105.

The amount of the sacrificial metal alkyl compound that is introduced to the stream or equipment (e.g., via stream 108A, 108B, 108C, 108D, 108E, 108F, or combinations thereof) is based on a temperature of the process liquid in the reboiler 130. The stream or equipment to which the sacrificial metal alkyl compound is introduced is any of those described hereinabove. The process can further include separating the nonreactive solid product from the aromatic compound (e.g., in separator 210 or filter 310, or both the separator 210 and the filer 310); after separating, introducing the aromatic compound and hydrogen into the liquid phase hydrogenation reactor 120; and reacting the aromatic compound with the hydrogen in a presence of a homogeneous hydrogenation catalyst in the liquid phase hydrogenation reactor 120 to form a cycloalkane compound.

In aspects where the separator 210 comprises a static mixer, adsorbent bed, a hydrocyclone, or combinations thereof, separating can include flowing the purified aromatic hydrocarbon stream 105 through the separator 210 to produce a hydrogenation reactor feed stream 107 comprising the aromatic compound, wherein the hydrogenation reactor feed stream 107 is connected to an inlet of the liquid phase hydrogenation reactor 120.

In aspects separating can include flowing the purified aromatic hydrocarbon stream through the filter 310 to produce a hydrogenation reactor feed stream (the permeate stream 109) comprising the aromatic compound, wherein the hydrogenation reactor feed stream (the permeate stream 109) is connected to an inlet of the liquid phase hydrogenation reactor 120.

The process can further include introducing the aromatic compound and hydrogen into the liquid phase hydrogenation reactor 120; reacting the aromatic compound with the hydrogen in a presence of a homogeneous hydrogenation catalyst in the liquid phase hydrogenation reactor 120 to form a cycloalkane compound; withdrawing a portion of a liquid reaction medium comprising liquid phase reaction components and solid particulates from the liquid phase hydrogenation reactor 120; flowing the portion of the reaction medium that is withdrawn from the liquid phase hydrogenation reactor 120 through the liquid cooling loop 140; removing part of the portion from the liquid cooling loop 140; filtering the part (e.g., with filter 430) to produce a permeate comprising a portion of the liquid phase reaction components and a retentate comprising the solid particulates; and recycling the permeate to the liquid cooling loop 140 or directly to the liquid phase hydrogenation reactor 120.

### Additional Description

Aspect 1. A process comprising: introducing a sacrificial metal alkyl compound to a stream or equipment comprising an aromatic compound and a catalyst deactivation compound, wherein a location of the stream or equipment is upstream of a liquid phase hydrogenation reactor, wherein the location is not in a liquid cooling loop of the liquid phase hydrogenation reactor; and reacting the sacrificial metal alkyl compound with the catalyst deactivation compound in the stream or equipment to form a nonreactive solid product.

Aspect 2. The process of Aspect 1, further comprising: prior to introducing the sacrificial metal alkyl compound, distilling a crude aromatic mixture comprising the aromatic compound, the catalyst deactivation compound, and water into an overhead product comprising the water and a bottoms product comprising the aromatic compound and the catalyst deactivation compound; and reboiling, in a reboiler, the bottoms product to form a recycle stream and a purified aromatic hydrocarbon stream comprising the aromatic compound, wherein aromatic compound is introduced to the liquid phase hydrogenation reactor via the purified aromatic hydrocarbon stream.

Aspect 3. The process of Aspect 2, wherein an amount of the sacrificial metal alkyl compound that is introduced to the stream or equipment is i) based on a temperature of process liquid in the reboiler, ii) based on a temperature of process liquid in the purified aromatic hydrocarbon stream, or iii) both i) and ii).

Aspect 4. The process of any one of Aspects 1 to 3, wherein the stream or equipment to which the sacrificial metal alkyl compound is introduced is the reboiler, the recycle stream, the purified aromatic hydrocarbon stream, or a combination thereof.

Aspect 5. The process of any one of Aspects 1 to 4, wherein the purified aromatic hydrocarbon stream is connected to the liquid phase hydrogenation reactor.

Aspect 6. The process of any one of Aspects 1 to 5, wherein the purified aromatic hydrocarbon stream contains less than 20 ppmw of the water based on a total weight of the purified aromatic hydrocarbon stream.

Aspect 7. The process of any one of Aspects 1 to 6, further comprising: separating the nonreactive solid product from the aromatic compound; after separating, introducing the aromatic compound and hydrogen into the liquid phase hydrogenation reactor; and reacting the aromatic compound with the hydrogen in a presence of a homogeneous hydrogenation catalyst in the liquid phase hydrogenation reactor to form a cycloalkane compound.

Aspect 8. The process of Aspect 7, wherein separating comprises: flowing the purified aromatic hydrocarbon stream through a static mixer, adsorbent bed, a hydrocyclone, or a combination thereof, to produce a hydrogenation reactor feed stream comprising the aromatic compound, wherein the hydrogenation reactor feed stream is connected to the liquid phase hydrogenation reactor.

Aspect 9. The process of Aspect 7 or 8, wherein separating comprises: flowing the purified aromatic hydrocarbon stream through a filter to produce a hydrogenation reactor feed stream comprising the aromatic compound, wherein the hydrogenation reactor feed stream is connected to the liquid phase hydrogenation reactor.

Aspect 10. The process of any one of Aspects 1 to 9, further comprising: introducing the aromatic compound and hydrogen into the liquid phase hydrogenation reactor; reacting the aromatic compound with the hydrogen in a presence of a homogeneous hydrogenation catalyst in the liquid phase hydrogenation reactor to form a cycloalkane compound; withdrawing a portion of a liquid reaction medium comprising liquid phase reaction components and solid particulates from the liquid phase hydrogenation reactor; flowing the portion of the liquid reaction medium that is withdrawn from the liquid phase hydrogenation reactor through the liquid cooling loop; removing part of the portion from the liquid cooling loop; filtering the part to produce a permeate comprising a portion of the liquid phase reaction components and a retentate comprising the solid particulates; and recycling the permeate to the liquid cooling loop or directly to the liquid phase hydrogenation reactor.

Aspect 11. The process of Aspect 10, wherein the solid particulates comprise the nonreactive solid product.

Aspect 12. The process of any one of Aspects 1 to 11, wherein the catalyst deactivation compound comprises an oxygen-containing compound, a sulfur-containing compound, a halide-containing compound, or combinations thereof, which is/are reactive with one or more Group 10 metals as defined by International Union of Pure and Applied Chemistry (IUPAC).

Aspect 13. The process of any one of Aspects 1 to 12, wherein the sacrificial metal alkyl compound has a formula R₃M, where R is an aliphatic hydrocarbon group having from 1 to 30 carbon atoms, wherein M is aluminum, zinc, lithium, or combinations thereof.

Aspect 14. The process of any one of Aspects 1 to 13, wherein the aromatic compound is benzene or toluene.

Aspect 15. A system for liquid phase hydrogenation of an aromatic compound, the system comprising: a distillation column operable to separate a crude aromatic mixture comprising the aromatic compound, a catalyst deactivation compound, and water into an overhead stream comprising water and a bottoms stream comprising the aromatic compound and the catalyst deactivation compound; a reboiler operable to heat the bottoms stream to form recycle stream and a purified aromatic hydrocarbon stream; a sacrificial metal alkyl stream comprising a metal alkyl compound connected to the reboiler, to the recycle stream, to the purified aromatic hydrocarbon stream, or a combination thereof; a liquid phase hydrogenation reactor operable to react the aromatic compound with hydrogen in a presence of a homogeneous hydrogenation catalyst to form a cycloalkane compound; and a liquid cooling loop operable to receive a portion of a liquid reaction medium comprising liquid phase reaction components and solid particulates and to cool the portion of the liquid reaction medium prior to recycling the cooled liquid reaction medium to the liquid phase hydrogenation reactor.

Aspect 16. The system of Aspect 15, further comprising: a separator having an inlet connected to the purified aromatic hydrocarbon stream and an outlet connected to an inlet of the liquid phase hydrogenation reactor, wherein the separator is operable to separate a nonreactive solid product from the aromatic compound.

Aspect 17. The system of Aspect 16, wherein the separator comprises a static mixer, an adsorbent bed, a hydrocyclone, or a combination thereof.

Aspect 18. The system of Aspect 16 or 17, wherein the separator comprises a filter.

Aspect 19. The system of any one of Aspects 15 to 18, further comprising: a slip stream connected to the liquid cooling loop and operable to receive part of the portion of the liquid reaction medium; a filter having an inlet fluidly coupled to the slip stream and operable to separate the slip stream into a permeate comprising a portion of the liquid phase reaction components and a retentate comprising the solid particulates; and a permeate stream connected to the filter and to the liquid cooling loop, and operable to flow the permeate from the filter to the liquid cooling loop.

Aspect 20. The system of any one of Aspects 15 to 19, further comprising: a hydrogen feed line connected to the purified aromatic hydrocarbon stream, wherein the sacrificial metal alkyl stream is connected to the hydrogen feed line.

Although the present disclosure and its advantages have been described in detail, it should be understood that various changes, substitutions, and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A process comprising:
introducing a sacrificial metal alkyl compound to a stream or equipment comprising an aromatic compound and a catalyst deactivation compound, wherein a location of the stream or equipment is upstream of a liquid phase hydrogenation reactor, wherein the location is not in a liquid cooling loop of the liquid phase hydrogenation reactor; and
reacting the sacrificial metal alkyl compound with the catalyst deactivation compound in the stream or equipment to form a nonreactive solid product.

2. The process of claim 1, further comprising:
prior to introducing the sacrificial metal alkyl compound, distilling a crude aromatic mixture comprising the aromatic compound, the catalyst deactivation compound, and water into an overhead product comprising the water and a bottoms product comprising the aromatic compound and the catalyst deactivation compound; and
reboiling, in a reboiler, the bottoms product to form a recycle stream and a purified aromatic hydrocarbon stream comprising the aromatic compound,
wherein aromatic compound is introduced to the liquid phase hydrogenation reactor via the purified aromatic hydrocarbon stream.

3. The process of claim 2, wherein an amount of the sacrificial metal alkyl compound that is introduced to the stream or equipment is based on a temperature of process liquid in the reboiler.

4. The process of claim 2, wherein the stream or equipment to which the sacrificial metal alkyl compound is introduced is the reboiler, the recycle stream, the purified aromatic hydrocarbon stream, or a combination thereof.

5. The process of claim 4, wherein the purified aromatic hydrocarbon stream is connected to the liquid phase hydrogenation reactor.

6. The process of claim 2, wherein the purified aromatic hydrocarbon stream contains less than 20 ppmw of the water based on a total weight of the purified aromatic hydrocarbon stream.

7. The process of claim 2, further comprising:
separating the nonreactive solid product from the aromatic compound;
after separating, introducing the aromatic compound and hydrogen into the liquid phase hydrogenation reactor; and
reacting the aromatic compound with the hydrogen in a presence of a homogeneous hydrogenation catalyst in the liquid phase hydrogenation reactor to form a cycloalkane compound.

8. The process of claim 7, wherein separating comprises:
flowing the purified aromatic hydrocarbon stream through a static mixer, adsorbent bed, a hydrocyclone, or a combination thereof, to produce a hydrogenation reactor feed stream comprising the aromatic compound,
wherein the hydrogenation reactor feed stream is connected to the liquid phase hydrogenation reactor.

9. The process of claim 7, wherein separating comprises:
flowing the purified aromatic hydrocarbon stream through a filter to produce a hydrogenation reactor feed stream comprising the aromatic compound,
wherein the hydrogenation reactor feed stream is connected to the liquid phase hydrogenation reactor.

10. The process of claim 1, further comprising:
introducing the aromatic compound and hydrogen into the liquid phase hydrogenation reactor;
reacting the aromatic compound with the hydrogen in a presence of a homogeneous hydrogenation catalyst in the liquid phase hydrogenation reactor to form a cycloalkane compound;
withdrawing a portion of a liquid reaction medium comprising liquid phase reaction components and solid particulates from the liquid phase hydrogenation reactor;
flowing the portion of the liquid reaction medium that is withdrawn from the liquid phase hydrogenation reactor through the liquid cooling loop;
removing part of the portion from the liquid cooling loop;
filtering the part to produce a permeate comprising a portion of the liquid phase reaction components and a retentate comprising the solid particulates; and
recycling the permeate to the liquid cooling loop or directly to the liquid phase hydrogenation reactor.

11. The process of claim 10, wherein the solid particulates comprise the nonreactive solid product.

12. The process of claim 1, wherein the catalyst deactivation compound comprises an oxygen-containing compound, a sulfur-containing compound, a halide-containing compound, or combinations thereof, which is/are reactive with one or more Group 10 metals as defined by International Union of Pure and Applied Chemistry (IUPAC).

13. The process of claim 1, wherein the sacrificial metal alkyl compound has a formula R₃M, where R is an aliphatic hydrocarbon group having from 1 to 30 carbon atoms, wherein M is aluminum, zinc, lithium, or combinations thereof.

14. The process of claim 1, wherein the aromatic compound is benzene or toluene.

15. A system for liquid phase hydrogenation of an aromatic compound, the system comprising:
a distillation column operable to separate a crude aromatic mixture comprising the aromatic compound, a catalyst deactivation compound, and water into an overhead stream comprising water and a bottoms stream comprising the aromatic compound and the catalyst deactivation compound;
a reboiler operable to heat the bottoms stream to form recycle stream and a purified aromatic hydrocarbon stream;
a sacrificial metal alkyl stream comprising a metal alkyl compound connected to the reboiler, to the recycle stream, to the purified aromatic hydrocarbon stream, or a combination thereof;
a liquid phase hydrogenation reactor operable to react the aromatic compound with hydrogen in a presence of a homogeneous hydrogenation catalyst to form a cycloalkane compound; and
a liquid cooling loop operable to receive a portion of a liquid reaction medium comprising liquid phase reaction components and solid particulates and to cool the portion of the liquid reaction medium prior to recycling the cooled liquid reaction medium to the liquid phase hydrogenation reactor.

16. The system of claim 15, further comprising:
a separator having an inlet connected to the purified aromatic hydrocarbon stream and an outlet connected to an inlet of the liquid phase hydrogenation reactor, wherein the separator is operable to separate a nonreactive solid product from the aromatic compound.

17. The system of claim 16, wherein the separator comprises a static mixer, an adsorbent bed, a hydrocyclone, or a combination thereof.

18. The system of claim 16, wherein the separator comprises a filter.

19. The system of claim 15, further comprising:
a slip stream connected to the liquid cooling loop and operable to receive part of the portion of the liquid reaction medium;
a filter having an inlet fluidly coupled to the slip stream and operable to separate the slip stream into a permeate comprising a portion of the liquid phase reaction components and a retentate comprising the solid particulates; and
a permeate stream connected to the filter and to the liquid cooling loop, and operable to flow the permeate from the filter to the liquid cooling loop.

20. The system of claim 15, further comprising:
a hydrogen feed line connected to the purified aromatic hydrocarbon stream, wherein the sacrificial metal alkyl stream is connected to the hydrogen feed line.
